(19) United States
(12) Patent Application Publication
(10) Pub. No.: US 2012/0190922 A1
(43) Pub. Date: Jul. 26, 2012

(54) ENDOSCOPE SYSTEM

(75) Inventor: Toshihiko KAKU, Kanagawa (JP)

(73) Assignee: Fujifilm Corporation, Tokyo (JP)

(21) Appl. No.: 13/323,568

(22) Filed: Dec. 12, 2011

(30) Foreign Application Priority Data
Jan. 24, 2011 (JP) 2011-011985

(51) Int. Cl. A61B 1/04

(52) U.S. Cl. 600/109

(57) ABSTRACT

An endoscope system is provided in which blood vessels (pits) are extracted from an image captured by an endoscope to perform pattern matching with vascular patterns (pit patterns) associated with the cancer invasion depth and operations such as enhanced display of blood vessels, suggestion of preferred imaging conditions and display for diagnosis support are performed based on the matching result. The endoscope system enables even a less-experienced doctor to correctly diagnose the cancer invasion depth.

### BACKGROUND OF THE INVENTION

The present invention relates to an endoscope system suitable to the diagnosis using, for example, microvessels in the superficial layer. The invention more specifically relates to an endoscope system with which even a less-experienced doctor can properly diagnose the cancer invasion depth from microvessels in the superficial layer.

In recent years, use is made of an endoscope system capable of so-called special light observation in which a mucosal tissue of a living body is irradiated with specific narrow wavelength band light (narrowband light) as observation light to obtain tissue information at a desired depth of the tissue of the living body.

The special light observation can simply visualize biological information which is not obtainable from the normal observation image, such as the microstructure in the superficial layer of a neovascular vessel formed in a mucosal layer or a submucosal layer, or an enhanced lesion. For example when a cancer lesion is to be observed, a mucosal tissue is irradiated with blue (B) narrowband light suitable to the observation of the tissue in the superficial layer and green (G) narrowband light suitable to the observation of the tissue in the middle and superficial layers, whereby the state of the microvessels and microstructure in the superficial layer of the tissue can be more precisely observed and the lesion can be therefore more correctly diagnosed.

The endoscope system as described in JP 3559755 B and JP 3607857 B is known as one having the function of special light observation.

The endoscope system uses a light source device which includes a light source for emitting white light and a rotary filter having an R filter for converting white light into red (R) light, a G filter for converting white light into green (G) light and a B filter for converting white light into blue (B) light, and an endoscope which captures images with an monochrome CCD sensor.

In the endoscope system, the rotary filter of the light source device is rotated to sequentially insert the color filters in the optical path to generate in a time-sharing manner R light, G light and B light, which are then sequentially supplied to the endoscope as observation light.

On the other hand, the endoscope irradiates an observation site with the observation light (R light, G light and B light) supplied from the light source device and sequentially captures an R image, a G image and a B image of the observation site with an imaging element in a so-called frame sequential manner.

The endoscope system uses the rotary filter of a double structure including a first set of outside filters and a second set of inside filters.

The first set of outside filters are filters for use in the normal light observation which have such spectral characteristics that the wavelength ranges of the respective colors overlap each other. On the other hand, the second set of inside filters are filters for use in the special light observation which have such discrete narrowband spectral characteristics that the wavelength ranges of the respective colors are separated from each other.

Therefore, this endoscope system is capable of both of the normal light observation and the special light observation by shifting the rotational axis of the rotary filter so that the first set of filters may act on the optical path when the normal light observation is performed and the second set of filters may act on the optical path when the special light observation is performed.

### SUMMARY OF THE INVENTION

As described above, the special light observation using narrowband light enables the state of the microvessels and microstructure in the superficial layer of the tissue to be more precisely observed. Therefore, the cancer lesion can be more correctly diagnosed.

In the cancer diagnosis, the cancer invasion depth is diagnosed for the subsequent proper treatment. With the recent progress in the endoscopic diagnosis, the cancer invasion depth can be diagnosed from the blood stream in microvessels of the superficial layer observed by the endoscope.

However, the blood stream in the microvessels of the superficial layer is complicated and varies with the cancer invasion depth and the site of the affected area such as esophagus or stomach.

Therefore, there arises a problem that doctors have difficulty in correctly diagnosing the cancer invasion depth unless they have sufficient experience in the endoscopic diagnosis of cancer.

An object of the invention is to solve the foregoing prior art problems and to provide an endoscope system with which even a less-experienced doctor can properly diagnose the cancer invasion depth from endoscopic images of microvessels in the superficial layer.

In order to achieve the above object, the present invention provides an endoscope system including: an endoscope having an imaging device for photoelectrically capturing an image; a light source device for supplying to the endoscope observation light used for capturing the image in the endoscope; an image processing device for processing the image captured by the endoscope to generate a display image; and a monitor for displaying the display image generated in the image processing device, wherein the image processing device comprises a storage section for storing at least one of vascular pattern classification including vascular patterns associated with a disease invasion depth and pit pattern classification including pit patterns associated with the disease invasion depth; an extracting section for extracting at least one of blood vessels and pits from he image captured by the endoscope; a matching section for performing pattern matching between at least one of the blood vessels and pits extracted by the extracting section and at least one of the vascular patterns in the vascular pattern classification and the pit patterns in the pit pattern classification which are stored in the storage section; and a diagnosis support section for receiving a result of the pattern matching from the matching section and causing at least one of the endoscope, the light source device, the monitor and the image processing device to perform support operations for diagnosis support based on the result of the pattern matching.

In the endoscope system of the invention, the light source device preferably has a function of supplying narrowband light for special light observation to the endoscope.

The support operations preferably include at least one of adjustment of imaging conditions, adjustment of image processing performed in the image processing device and the diagnosis support through display on the monitor.

The adjustment of the imaging conditions preferably includes at least one of changing an imaging magnification in the endoscope, changing an f-number in the endoscope, adjusting quantities of the observation light from the light source device, and adjusting a spectral distribution of the observation light from the light source device.

The adjustment of the image processing performed in the image processing device preferably includes at least one of enhancement of a blood vessel matching with a vascular pattern in the vascular pattern classification stored in the storage section and enhancement of a pit matching with a pit pattern in the pit pattern classification stored in the storage section.

The diagnosis support through the display on the monitor is preferably the display of the result of the pattern matching in the matching section.

The diagnosis support through the display on the monitor is preferably the display prompting an operator to perform at least one of changing an imaging magnification in the endoscope, changing an f-number in the endoscope, adjusting quantities of the observation light from the light source device, and adjusting a spectral distribution of the observation light from the light source device.

A degree of the support operations is preferably changed according to at least one of the disease invasion depth in a vascular pattern which matched with a blood vessel and the disease invasion depth in a pit pattern which matched with a pit.

The endoscope system preferably further comprises a selector for selecting execution of extraction in the extracting section and execution of the pattern matching in the matching section.

The matching section preferably performs the pattern matching according to at least one of an amount of the blood vessels and an amount of the pits extracted by the extracting section.

The storage section preferably stores at least one of the vascular pattern classification and the pit pattern classification for each of at least two diagnostic sites.

The endoscope system preferably further comprises an indicator for indicating a site to be observed with the endoscope.

The endoscope system of the invention configured as described above extracts blood vessels or the like from a special light observation image using narrowband light as observation light, performs pattern matching of the blood vessels with vascular patterns associated with the cancer invasion depth, and performs the following diagnosis support based on the result of the pattern matching: output of the cancer invasion depth, enhanced display of blood vessels deemed to be affected, and changes in the imaging conditions to obtain an image capable of more suitable observation.

Therefore, according to the endoscope system of the invention, even a doctor with less experience in the endoscopic cancer diagnosis can properly diagnose the cancer invasion depth from endoscopic observation images.

### BRIEF DESCRIPTION OF THE DRAWINGS

FIG. 1 is a conceptual perspective view showing an embodiment of an endoscope system of the invention.

FIG. 2 is a conceptual block diagram showing the configuration of the endoscope system shown in FIG. 1.

FIG. 3 is a conceptual diagram showing spectral characteristics of observation light in the endoscope system shown in FIG. 1.

FIG. 4 is a conceptual block diagram showing a processor of the endoscope system shown in FIG. 1.

FIG. 5 is a conceptual diagram showing a type of pattern classification stored in the endoscope system shown in FIG. 1.

FIG. 6 is a conceptual diagram showing another type of pattern classification stored in the endoscope system shown in FIG. 1.

FIG. 7 is a conceptual diagram showing still another type of pattern classification stored in the endoscope system shown in FIG. 1.

FIG. 8 is a conceptual diagram showing yet another type of pattern classification stored in the endoscope system shown in FIG. 1.

### DETAILED DESCRIPTION OF THE INVENTION

On the following pages, the endoscope system of the invention is described in detail with reference to the preferred embodiments illustrated in the accompanying drawings.

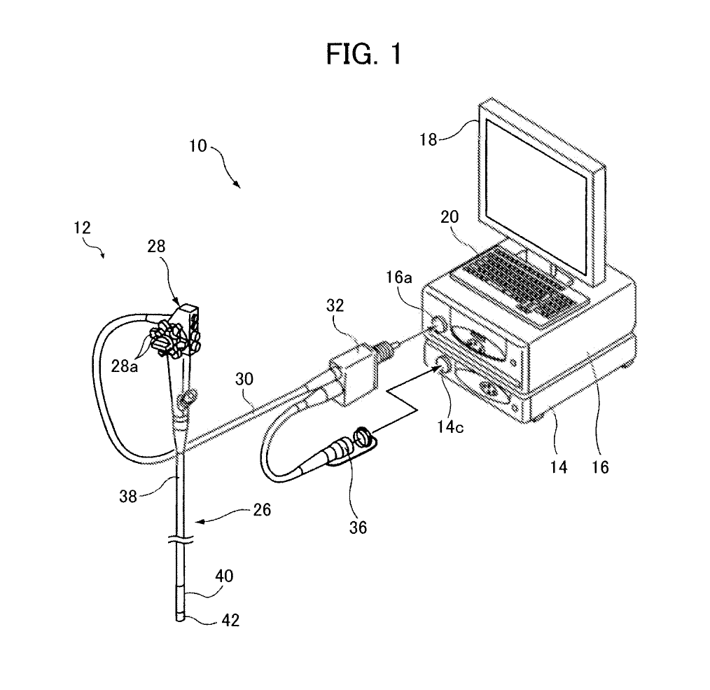

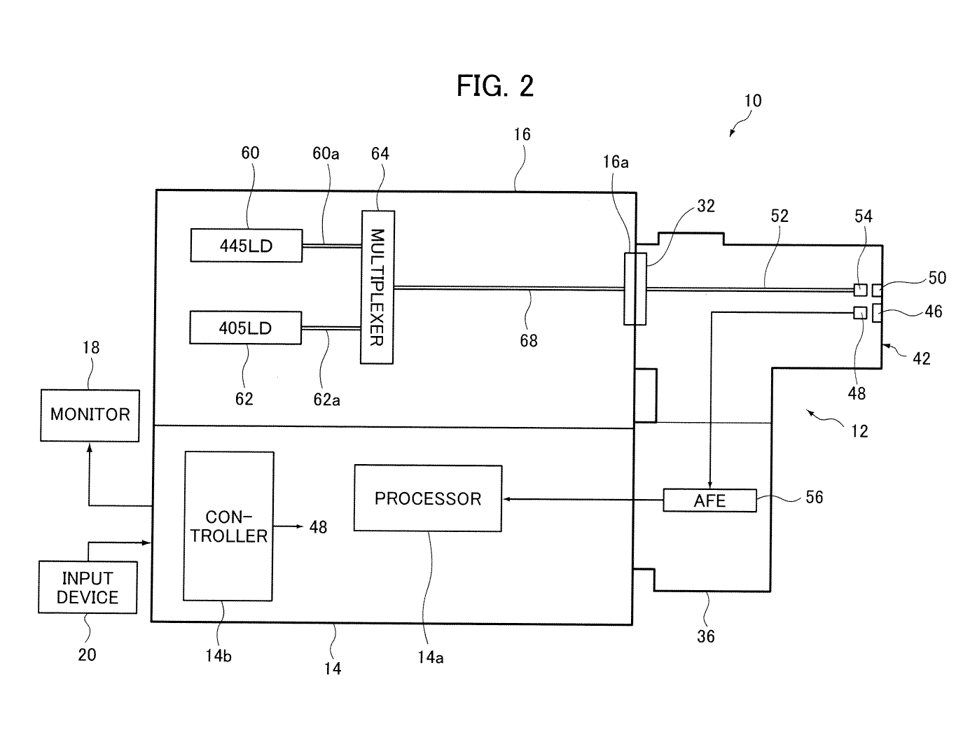

FIG. 1 is a conceptual perspective view showing an embodiment of the endoscope system of the invention and FIG. 2 conceptually shows the configuration of the endoscope system shown in FIG. 1.

The illustrated endoscope system 10 includes, for example, an endoscope 12, a processing device 14 for processing an image captured by the endoscope 12 and a light source device 16 for supplying observation light (illumination light) for use in observation and image capture using the endoscope 12.

The processing device 14 is connected to a monitor 18 for displaying an image captured by the endoscope and an input device 20 for inputting various instructions. The processing device 14 may further be connected to a printer (recording unit) for outputting an image captured by the endoscope as a hard copy.

As shown in FIG. 2, the endoscope 12 is an electronic endoscope which photoelectrically captures an image using an imaging device such as a CCD sensor 48. As in a common endoscope, the endoscope 12 includes an insertion section 26, an operating section 28, a universal cord 30, a connector 32 and a video connector 36.

During the observation (diagnosis), the endoscope 12 is used with the video connector 36 and the connector 32 connected to a connecting portion 14c of the processing device 14 and a connecting portion 16a of the light source device 16, respectively. As in a common endoscope, the connector 32 is connected to a suction means and an air supply means for the suction from and the air supply to the site to be observed, and a water supply means for the water injection on the site to be observed.

As in a common endoscope, the insertion section 26 of the endoscope 12 includes a long flexible portion 38 on the proximal side, a distal scope portion (endoscope distal portion) 42 provided with the COD sensor 48 and so on, and a bending portion (angle portion) 40 located between the flexible portion 38 and the scope portion 42. The operating section 28 includes manipulation knobs 28a for bending the bending portion 40.

As schematically shown in FIG. 2, the scope portion 42 is provided with an imaging lens 46, the CCD sensor 48, an illumination lens 50, an optical fiber 52, a fluorescent body 54 and a cover glass (not shown) for protecting the lenses and the like.

Although not shown, the endoscope 12 is also provided with a forceps channel and a forceps port for inserting various treatment tools such as a forceps, and air supply/water supply channels and air supply/water supply ports for use in suction, air supply and water supply.

The forceps channel extends from the scope portion 42 through the bending portion 40 and the flexible portion 38 to communicate with a forceps insertion port provided in the operating section 28. The air supply/water supply channels extend from the scope portion 42 through the bending portion 40, the flexible portion 38, the operating section 28 and the universal cord 30 to communicate with connecting portions with the suction means, the air supply means and the water supply means in the connector 32.

The optical fiber 52 extends through the bending portion 40, the flexible portion 38, the operating section 28 and the universal cord 30 and terminated by the connector 32 which is connected to the light source device 16.

Observation light (excitation light) emitted from the light source device 16 to be described later enters the optical fiber 52 through the connector 32 and propagates through the optical fiber 52. In the scope portion 42, the light enters the fluorescent body 54 from the distal end of the optical fiber 52.

The fluorescent body 54 contains a plurality of phosphors which absorb part of B light, excite and emit green to yellow light.

Although described later in detail, the light source device 16 emits B light (B laser beam) with a central wavelength of 445 nm as excitation light. The B light excites the fluorescent body 54, which emits green to yellow excitation light. The green to yellow excitation light is combined with part of the B light which was not absorbed in the fluorescent body 54 but passed therethrough to form pseudo white light, which is used as observation light in the normal light observation.

The fluorescent body 54 will be described later in further detail together with the light source device 16.

The light emitted by the excitation of the fluorescent body 54 and part of the B light (and optionally narrowband BV light to be described later) having passed through the fluorescent body 54 enters and passes through the illumination lens 50 to be irradiated on an observation site.

The observation site having received the observation light (reflected light from the living body) is imaged through the imaging lens 46 on the light-receiving surface of the CCD sensor 48.

The CCD sensor 48 is a common color CCD sensor which is used in a common endoscope or digital camera and separates incident light into R (red) light, G (green) light and B (blue) light of three primary colors for simultaneous photometry. Therefore, the CCD sensor 48 outputs signals of an R image, a G image and a B image.

In the practice of the invention, the imaging device is not limited to the CCD sensor 48 and various known imaging devices such as a CMOS image sensor may be used.

Output signals from the CCD sensor 48 are sent on signal lines from the scope portion 42 to the video connector 36 through the bending portion 40, the flexible portion 38, the operating section 28, the universal cord 30 and the connector 32.

In the illustrated embodiment, an AFE (Analog Front End) board 56 is disposed in the video connector 36.

The AFE board 56 includes, for example, a correlated double sampling circuit, an amplifier (automatic gain control circuit) and an A/D converter. In the AFE board 56, the output signals from the CCD sensor 48 are subjected to noise removal by correlated double sampling, amplification in the amplifier and conversion of analog signals into digital signals in the A/D converter, and then outputted to the processing device 14 (more specifically to a DSP 72 to be described later) as digital image signals.

In the endoscope system of the invention, these processing steps may be performed in the connector 32 or the processing device 14 instead of the video connector 36.

As described above, the connector 32 of the endoscope 12 in the endoscope system 10 is connected to the connecting portion 16a of the light source device 16.

The light source device 16 supplies the endoscope 12 with observation light for the internal observation of a living body. As described above, the observation light (excitation light) supplied from the light source device 16 toward the endoscope 12 enters the optical fiber 52 through the connector 32 and propagates therethrough to be irradiated on the observation site through the scope portion 42.

As schematically shown in FIG. 2, the light source device 16 of the endoscope system 10 includes a 445LD 60, a 405LD 62, optical fibers 60a and 62a, a (optical) multiplexer 64 and an optical fiber 68.

The 445LD 60 is a light source emitting B laser beams with a central wavelength of 445 nm. On the other hand, the 405LD 62 is a light source emitting blue-violet (BV) laser beams with a central wavelength of 405 nm (light source emitting narrowband BV light). Both the light sources will be described in detail later.

The B light emitted from the 445LD 60 is propagated through the optical fiber 60a and the BV light emitted from the 405LD 62 is propagated through the optical fiber 62a and the B light and BV light are multiplexed into a single beam by the multiplexer 64.

The B light and BV light are multiplexed into the single beam, which is propagated through the optical fiber 68, is supplied through the connecting portion 16a to the connector 32 of the endoscope 12, and enters and is propagated through the optical fiber 52 to exit from the distal end of the optical fiber 52.

As described above, in the endoscope 12, the fluorescent body 54 is disposed at the distal end of the optical fiber 52. The fluorescent body 54 contains a plurality of kinds of phosphors (e.g., YAG phosphor and BAM (BaMgAl₁₀O₁₇) phosphor) which absorb part of B light, excite and emit green to yellow light. As described above, the green to yellow light emitted by the excitation of the fluorescent body having absorbed the B light as excitation light is combined with part of the B light which was not absorbed in the fluorescent body 24 but passed therethrough to form pseudo white light.

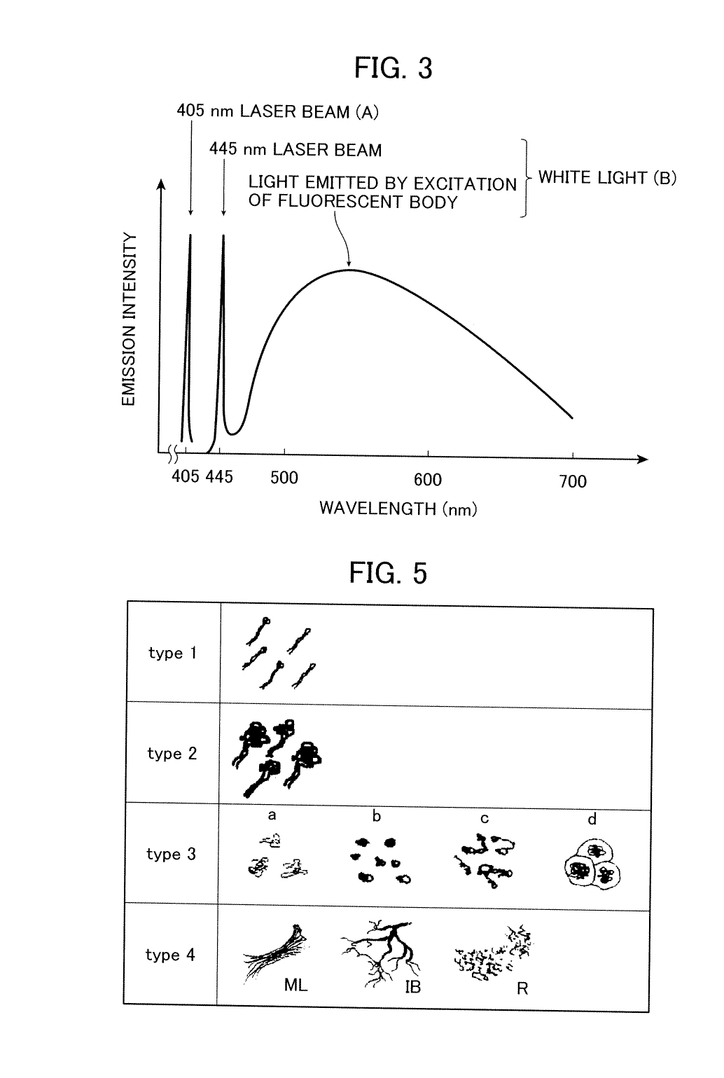

FIG. 3 shows spectral characteristics of the BV light emitted from the 405LD 62, the B light emitted from the 445LD 60 and the light emitted from the fluorescent body 24 excited by the B light (445 nm laser beam) (emission spectrum from the fluorescent body 24 excited by the B light).

As shown in FIG. 3, the BV light emitted from the 405LD 62 is narrowband BV light represented by an emission line with a central wavelength of 405 nm (profile A). The BV light is hardly absorbed in the fluorescent body 54 and passes therethrough.

On the other hand, the B light emitted from the 445LD 60 is represented by an emission line with a central wavelength of 445 nm. In addition, the light emitted from the fluorescent body 24 excited by the B light has a spectral intensity distribution which shows an increase in emission intensity in a wavelength range of about 450 nm to about 700 nm.

Therefore, when the 445LD 60 is only turned on in the light source device 16, the light emitted by the excitation of the fluorescent body 24 is combined with part of the B light from the 445LD 60 which was not absorbed in the fluorescent body 24 to form pseudo white light (profile B), which enables the normal light observation using white light as the observation light to be performed.

When both of the 445LD 60 and the 405LD 62 are turned on, narrowband BV light shown by profile A is added to the observation light, thus enabling imaging with special light.

In other words, in the light source device 16, the normal light observation using white light as observation light is possible when the 445LD 60 is turned on and the 405LD 62 is turned off, and the special light observation using white light and narrowband BV light as observation light is possible when the 445LD 60 and the 405LD 62 are both turned on.

In addition, normal light observation and special light observation can be performed substantially at the same time by turning on and off the 405LD 62 at predetermined intervals with the 445LD 60 kept turned on.

This point will be described in detail later.

The observation site having received the observation light from the scope portion 42 of the endoscope 12 is imaged by the CCD sensor 48.

As described above, the image captured by the CCD sensor 48 (output signals from the CCD sensor 48) is subjected to processing such as A/D conversion in the AFE board 56 and supplied to the processing device 16 as digital image signals (image data/image information).

The processing device 14 subjects the image signals supplied (outputted) from the endoscope 12 (the image signals are hereinafter also referred to simply as an “image”) to predetermined processing so that the monitor 18 displays them as an image captured by the endoscope 12 and also controls the endoscope system 10. The processing device 14 includes an image processor 14a and a controller 14b for controlling the whole of the endoscope system 10 including the processing device 14.

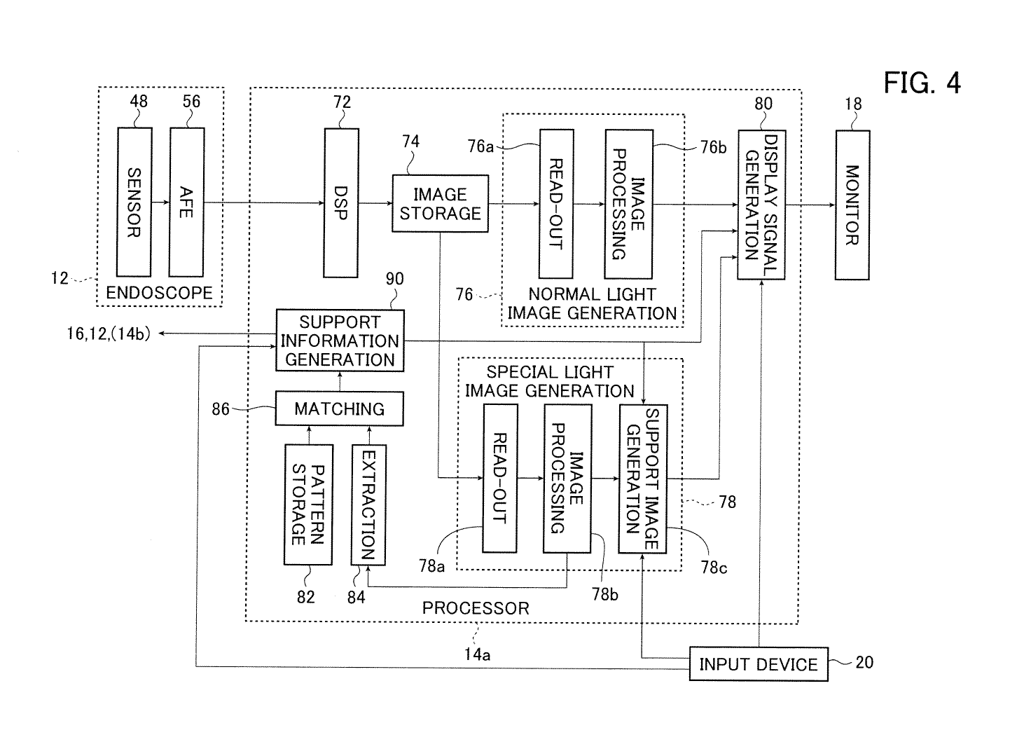

FIG. 4 is a conceptual block diagram showing the image processor 14a of the processing device 14.

As shown in FIG. 4, the image processor 14a includes the DSP 72, an image storage section 74, a normal light image generating section 76, a special light image generating section 78, a display signal generating section 80, a pattern storage section 82, an extracting section 84, a matching section 86 and a support information generating section 90.

In the processing device 14, images (R image, G image and B image) from the endoscope 12 are supplied to the DSP 72.

The DSP 72 is a known type of DSP (Digital Signal Processor), where the supplied images are subjected to predetermined processing steps such as gamma correction and color correction. The processed images are then stored in a predetermined region of the image storage section (memory) 74.

Once the images are stored in the image storage section 74, the normal light image generating section 76 reads out the R, G and B images from the image storage section 74 to generate a normal light observation image and the special light image generating section 78 reads out the B image (image formed with narrowband BV light) and the G image from the image storage section 74 to generate a special light observation image.

More specifically, in the light source device 16, the 405LD 62 is turned on and off at predetermined intervals (or high power lighting and low power lighting are alternately repeated) with the 445LD 60 kept turned on or continuously turned on with a predetermined quantity of light under the control of the controller 14b. On the other hand, the image is captured by the CCD sensor 48 of the endoscope 12 in synchronization with the on/off status of the 405LD 62.

As described above, the B light from the 445LD 60 enters and excites the fluorescent body 54, which emits green to yellow excitation light. Part of the B light which was not absorbed in the fluorescent body 24 but passed therethrough is combined with the green to yellow excitation light to form pseudo white light. The narrowband BV light emitted from the 405LD 62 is hardly absorbed in the fluorescent body 54 and passes therethrough, and therefore the white light and the narrowband BV light (narrowband B light) are used as observation light when the 405LD 62 is turned on.

Therefore, the normal light image generating section 76 reads out the R image, G image and B image from images stored in the image storage section 74 when the 405LD 62 is turned off (at the time of low power lighting) to enable the normal light observation image using the pseudo white light for the observation to be obtained. The special light image generating section 78 reads out the G image and B image from images stored in the image storage section 74 when the 405LD 62 is turned on (at the time of high power lighting) to enable the special light observation image using the narrowband BV light and G light for the observation to be obtained.

In other words, according to the illustrated endoscope system 10, the 405LD 62 is turned on and off at predetermined intervals, imaging is performed with the CCD sensor 48 in synchronization with the on/off status and the normal light image generating section 76 and the special light image generating section 78 read out images in synchronization with the imaging period to enable the simultaneously or substantially simultaneously captured normal light observation image and special light observation image to be obtained.

In the endoscope system 10, the normal light observation image can only be obtained when the 445LD 60 is continuously turned on and the 405LD 62 is continuously turned off, whereas the special light observation image can only be obtained when the 445LD 60 and the 405LD 62 are both continuously turned on.

The normal light image generating section 76 includes a read-out portion 76a and an image processing portion 76b.

The read-out portion 76a is a portion in which the R, G and B images are read out, as described above, from images stored in the image storage section 74 when the 405LD 62 is turned off and supplied to the image processing portion 76b.

The image processing portion 76b subjects the R, G and B images read out by the read-out portion 76a to processing with a 3×3 matrix, gradation conversion, processing with a three-dimensional LUT or other color conversion processing; color enhancement for giving a color difference between a blood vessel and a mucous membrane on the screen by enhancing in a direction in which the color difference between the blood vessel and the mucous membrane is to be more accentuated than the average colors of the image so that the blood vessel can be more easily seen; and image structure enhancement such as sharpening and edge enhancement, and supplies the processed image to the display signal generating section 80 as the normal light observation image.

The special light image generating section 78 includes a read-out portion 78a, an image processing portion 78b and a support image generating portion 78c.

The read-out portion 78a is a portion in which the B image (i.e., image captured with the narrowband BV light; hereinafter referred to as “narrowband BV image” for the sake of convenience) and the G image are read out, as described above, from images stored in the image storage section 74 when the 405LD 62 is turned on (at the time of high power lighting) and supplied to the image processing portion 78b.

The image processing portion 78b is a portion in which the narrowband BV image and the G image are processed to obtain the special light observation image.

On the displayed image (image outputted from the processing device 14), three sub-pixels of R, G and B make up one pixel. In the illustrated embodiment, however, the special light observation image is only obtained from the narrowband BV image and the G image. Therefore, the image processing portion 78b first allocates the G image to R pixels corresponding to the display and the narrowband BV image to G pixels and B pixels corresponding to the display to form an image in which three sub-pixels of R, G and B corresponding to the display make up one pixel.

The allocation of the narrowband BV image and the G image may be optionally preceded by image processing or correction such as multiplication of the images by a predetermined coefficient or processing with a predetermined LUT.

The image processing portion 78b subjects the images allocated to the R, G and B pixels to processing with a 3×3 matrix, gradation conversion, processing with a three-dimensional LUT or other color conversion processing; color enhancement for giving a color difference between a blood vessel and a mucous membrane on the screen by enhancing in a direction in which the color difference between the blood vessel and the mucous membrane is to be more accentuated than the average colors of the image so that the blood vessel can be more easily seen; and image structure enhancement such as sharpening and edge enhancement, thereby obtaining the special light observation image.

The special light observation image generated in the image processing portion 78b is supplied to the support image generating portion 78c and the extracting section 84 when an instruction for vascular or pit pattern matching of the special special light observation image which will be described later (diagnosis support by pattern matching) is issued.

Pattern matching with cancerous vascular patterns to be described later is preferably performed on special light observation images (narrowband light observation images) from which information on microvessels in the superficial layer or the like is easily obtained or microvessels in the superficial layer or the like is easily observed. However, the invention is not limited to this and the same processing may be performed on normal light observation images.

The support image generating portion 78c is a portion in which a blood vessel or pit which was detected by pattern matching of the special light observation image generated in the image processing portion 78b as matching with a cancerous vascular or pit pattern is enhanced in accordance with the instruction from the support information generating section 90 to be described later. The support image generating portion 78c will be described later in detail.

When an instruction is not issued for vascular or pit pattern matching of the special light observation image as in the case of withdrawing the insertion section 26 of the endoscope 12 after the end of the diagnosis, or when an instruction is not issued for image processing such as enhancement of a blood vessel or the like to be described later based on the result of the vascular or pit pattern matching, the special light observation image generated in the image processing portion 78b is directly supplied to the display signal generating section 80.

The extracting section 84 is a section in which, when an instruction is issued for matching with vascular or pit patterns, the special light observation image generated in the image processing portion 78b is analyzed to extract blood vessels and/or pits in the image (information of the blood vessels and/or information of the pits).

Therefore, when an instruction for pattern matching is not issued, no special light observation image is supplied to the extracting section 84.

The method of extracting blood vessels and pits is not particularly limited and various known methods can be used.

For example, the narrowband B light such as BV light is absorbed into blood vessels and the region where the narrowband B light is absorbed is darkened. Therefore, blood vessels or pits may be extracted by binarizing the image (image signals) with an appropriately set threshold based on changes of narrowband light in the blood vessels or the pits. Alternatively, the average value of the image is calculated and a region which is darker than the average value or a region having a different color may be extracted as the blood vessel or pit.

The image captured by the endoscope 12 has topographic features, a region obliquely irradiated with observation light and also a region where it is difficult to properly extract blood vessels or pits.

In such a case, the special light observation image may be appropriately divided into regions and blood vessel or pit extraction be performed in the respective regions so that the extraction results of the regions can be combined and used as the blood vessel or pit extraction result in the special light observation image.

In the endoscope system 10 of the invention, the portions to be extracted from the special light observation image are not limited to both of blood vessels and pits.

In other words, the extracting section 84 may extract only one of blood vessels and pits according to the pattern classification stored in the pattern storage section 82. Blood vessel extraction (vascular pattern matching) and pit extraction (pit pattern matching) may be provided as selectable modes so that one or both of them may be selected by the operation in the input device 20 or the operating section 28 of the endoscope 12.

The results of the extraction of blood vessels and pits from the special light observation image that was performed in the extracting section 84 are supplied to the matching section 86.

The matching section 86 is connected to the pattern storage section 82. The pattern storage section 82 is a section which stores vascular pattern classification including vascular patterns associated with the cancer invasion depth and/or pit pattern classification including pit patterns associated with the cancer invasion depth.

The matching section 86 is a section in which pattern matching is performed between the vascular patterns of the vascular pattern classification and/or the pit patterns of the pit pattern classification stored in the pattern storage section 82, and the blood vessels and/or pits extracted by the extracting section 84.

When cancer develops, a specific blood vessel which is not seen in a healthy site is generated (constructed) or a specific pit pattern which is not seen in a healthy site is generated in a microvessel in the superficial layer or IPCL (intraepithelial papillary capillary loop) of an affected area.

The shape of the blood vessel generated due to such a cancer (stream pattern of the neovascular vessel due to a cancer) and the shape of the pit (irregular pit-like structure due to a cancer) have a specific pattern according to the cancer invasion depth.

Therefore, the cancer invasion depth can be seen or diagnosed by storing vascular pattern classification including a plurality of vascular patterns associated with the cancer invasion depth or pit pattern classification including a plurality of pit patterns associated with the cancer invasion depth, extracting blood vessels or pits from an image captured by the endoscope, and performing the pattern matching between the extracted blood vessels and the vascular patterns associated with the invasion depth or between the extracted pits and the pit patterns associated with the invasion depth.

The vascular pattern and pit pattern associated with the cancer invasion depth differ depending on which area is affected with cancer, esophagus, large intestine or the like.

Therefore, the pattern storage section 82 preferably stores the vascular pattern classification and pit pattern classification for each of the sites to be diagnosed, such as esophagus, stomach, small intestine and large intestine (for at least two sites).

Various known types of cancer pattern classification specific to the diagnostic sites (observation sites) that were published in literatures and articles can be used for the vascular pattern classification and pit pattern classification (hereinafter collectively referred to simply as “pattern classification”) associated with the cancer invasion depth.

An example of the pattern classification in the case of esophageal cancer is the microvascular pattern classification illustrated in Magnifying Endoscopy with FICE for the Screening and Differential Diagnosis of Small Squamous Cell Carcinomas of the Esophagus, Stomach and Intestine, 2009;44: 1675-1687. This is shown in FIG. 5.

In the vascular pattern classification, type 1 is a vascular pattern in which thin and linear capillaries are observed in papillae. Type 2 is a vascular pattern in which blood vessels are extended, branched and enlarged, and have an increase in density but the structure is preserved, and the their arrangement is kept relatively regular. Type 3a is a broken filamentous vascular pattern; type 3b is a pattern of crushed vessels with red spots; type c is a vascular pattern in which the blood vessels in type 3b are elongated and bound together; and type 3d is a salmon roe-like vascular pattern in which spiral blood vessels are seen in papillary protrusions. Type 4ML is a multi-layered vascular pattern; type 4IB is an irregularly branched vascular pattern; and type 4R is a reticular vascular pattern.

In this microvessel pattern classification, the invasion depth of esophageal cancer successively increases from type 1 toward type 4.

In the microvascular pattern classification of esophageal cancer, type 4 may also include subclasses S such as S (up to 0.5 mm), M (more than 0.5 mm but up to 3 mm) and L (more than 3 mm) based on the avascular area (AVA) size.

Examples of the pattern classification of colorectal cancer include pit pattern classification (magnifying classification of pit-like structures) in Hiroshima University, Sano's capillary pattern (reticular capillary pattern; CP) classification; and vascular (microvessel-constructing) pattern classification (magnifying observation classification) considering the pit patterns in The Jikei University School of Medicine.

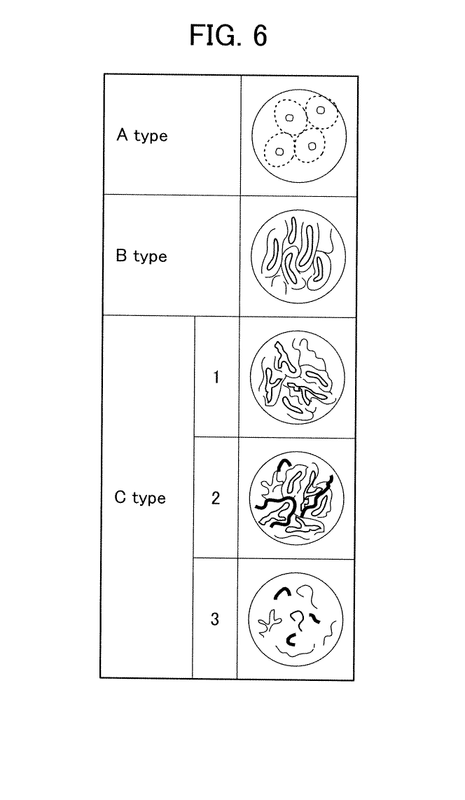

The pit pattern classification in Hiroshima University is the classification using the pit patterns shown in FIG. 6. This pattern classification additionally evaluates the microvascular patterns.

In this pit pattern classification, A type is a pit pattern having a normal color to brown color and no microvessel is visible. B type is a pit pattern in which a well-ordered clear pit-like structure is indirectly observed and which has a reticular microvessel architecture. C type 1 is a pit pattern indirectly having an irregular pit-like structure and blood vessels have a reticular architecture with relatively uniform thickness and distribution; C type 2 is a pit pattern indirectly having highly irregular pit-like structure and blood vessels have a reticular architecture with non-uniform thickness and distribution; and C type 3 is a pit pattern in which no irregular pit-like structure is clear and observable and blood vessels are irregular and have non-uniform thickness and distribution, an avascular area appears and scattered microvessel fragments are observed.

In this pit pattern classification, the invasion depth of colorectal cancer successively increases from A type toward C type and in C type, the invasion depth of colorectal cancers successively increases from 1 toward 3.

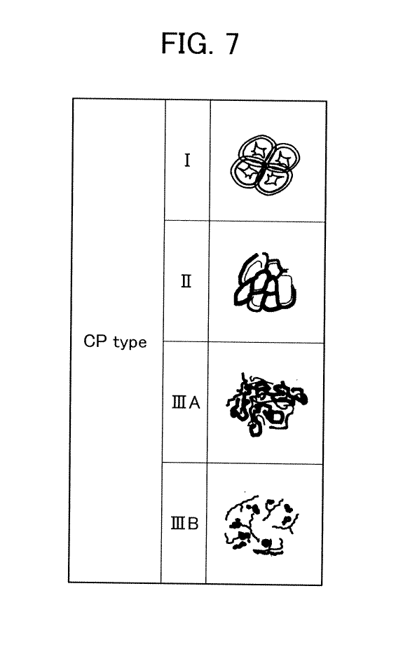

Sano's capillary pattern classification is the one as shown in FIG. 7.

In the capillary pattern classification, CP type I is a capillary pattern in which no microvessel can be confirmed. CP type II is a capillary pattern in which blood vessels surrounding the periphery of each gland duct can be observed and the blood vessel diameter is uniform. CP type IIIA is a capillary pattern in which blood vessels are not well ordered and irregular blood vessels are clearly seen. CP type IIIB is a capillary pattern in which blood vessels are not well ordered and irregular blood vessels are not clearly seen.

In this capillary pattern classification, the invasion depth of colorectal cancer successively increases from CP type I toward CP type III and in CP type III, the invasion depth is larger in B than A.

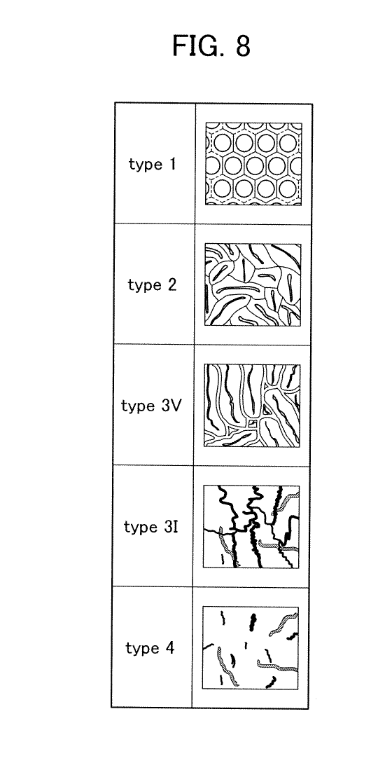

The vascular pattern classification in The Jikei University School of Medicine is the one as shown in FIG. 8.

In the vascular pattern classification, type 1 is a vascular pattern in which no blood stream is seen. Type 2 is a vascular pattern in which a slight increase in the blood vessel diameter is seen. Type 3 is a vascular pattern in which a marked increase in the vessel diameter is seen. More specifically, type 3V is a vascular pattern of a villous structure in which dilated vessels are regularly arranged along the gland duct interstitium and type 3I is a vascular pattern in which dilated vessels are irregularly arranged. Type 4 is a vascular pattern in which blood vessels are sparsely distributed and no blood stream can be seen.

In this vascular pattern classification, the invasion depth of colorectal cancer successively increases from type 1 toward type 4 and in type 3, the invasion depth is larger in I than V.

In the practice of the invention, a doctor skilled in the diagnosis of the cancer invasion depth using the endoscope system 10 may prepare vascular pattern classification or pit pattern classification from images actually used in the invasion depth diagnosis and store the prepared pattern classification in the pattern storage section 82 instead of using known types of pattern classification published in literatures and articles.

More specifically, the input device 20 may be provided with a registration button so that actual diagnostic images can be registered as vascular patterns or pit patterns making up the pattern classification.

An exemplary pattern classification method used when preparing pattern classification from actual diagnostic images involves storing images themselves as vascular patterns for the respective levels of invasion depth. Vascular or pit images extracted from diagnostic images may be stored as vascular patterns for the respective levels of invasion depth and used for the pattern classification. Specific regions (specific vascular structure and pit structure) may be selected from the extracted blood vessels or pits, stored as vascular patterns for the respective levels of invasion depth and used for the pattern classification. At least two of these methods may further be combined and used for the pattern classification.

In the preparation of the pattern classification from the actual diagnostic images, it is preferred to also store the scale factor, the quantities of observation light, the light quantity ratio (power ratio) between the 445LD 60 and the 405LD 62 in the light source device 16 and the imaging conditions of the CCD sensor 48 (e.g., electronic shutter speed) in association with the pattern classification.

Such pattern classification is generally prepared from enlarged observation images. On the other hand, in the actual diagnosis using the endoscope system 10, it is general to first perform screening at a low magnification (perform a broad observation as the scope portion 42 is moved), and enlarge, precisely examine and further make a qualitative diagnosis when an area deemed to be affected is detected by the screening.

Therefore, in each pattern classification, the vascular patterns and pit patterns are preferably stored at a plurality of magnifications set in the endoscope system 10 (e.g., 20×, 40×, 60× and 80×) so that pattern matching can be performed using images captured in the screening at low magnifications.

The vascular patterns and pit patterns at the respective magnifications may be produced by enlarging or reducing a pattern according to the imaging magnification used at the time of imaging the pattern and appropriately subjecting the enlarged or reduced pattern to processing such as sharpening, smoothing and brightness adjustment.

As described above, in such pattern classification, it is preferred to store pattern classification specific to the diagnostic sites such as esophagus, stomach and large intestine but a plurality of types of pattern classification may be stored in the pattern storage section 82 for each diagnostic site.

As described above, the matching section 86 is a section in which pattern matching (hereinafter referred to as “matching”) is performed to check whether blood vessels and/or pits extracted by the extracting section 84 match with the vascular patterns and/or pit patterns of the pattern classification stored in the pattern storage section 82.

The matching method used in the matching section 86 is not particularly limited and various known image matching methods (method of detecting matching images or degree of image matching) may be used, as exemplified by matching using vectors, matching using templates, matching using image characteristic quantities, and matching using two or more of these methods.

There is no particular limitation on the determination as to whether the blood vessels and/or pits extracted by the extracting section 84 match with the vascular patterns and/or pit patterns stored in the pattern storage section 82, and various methods can be used as long as whether the blood vessels (pits) match with the vascular patterns (pit patterns) can be determined.

For example, the degree of matching between a vascular or pit pattern and an extracted blood vessel or pit may be determined by a known method so that the former is deemed to match with the latter when the determined degree of matching is equal to or larger than an appropriately set threshold and the former is deemed not to match with the latter when the determined degree of matching is less than the threshold.

The matching method used in the matching section 86 is not limited to performing both of matching between blood vessels and vascular patterns (hereinafter referred to as “vascular matching”) and matching between pits and pit patterns (hereinafter referred to as “pit matching”).

More specifically, only one of vascular matching and pitch matching may be performed according to the extraction performed by the extraction section 84. Vascular matching and pitch matching may be provided as selectable modes as in the extraction performed in the extraction section 84.

The matching section 86 must select and read out pattern classification suitable to the diagnostic site from the pattern storage section 82 to perform vascular matching or pit matching.

The method of selecting the pattern classification in the matching section 86 is not particularly limited and various methods can be used.

For example, the input device 20 or the operating section 28 of the endoscope 12 may be provided with a selection switch for inputting a diagnostic site so that pattern classification corresponding to the diagnostic site can be read out in accordance with an instruction given with the selection switch.

In the common endoscope system 10, the processing device 14 can identify the model or individual device of the endoscope 12 at the time when the endoscope 12 is connected to the processing device 14 or the light source device 16. Based on this method, the endoscope 12 may read out the pattern classification corresponding to the diagnostic site in accordance with the result of the identification of the endoscope 12 made by the processing device 14.

Alternatively, in cases where the endoscope 12 is capable of determining the insertion length of the insertion section 26, the diagnostic site which is currently observed may be detected based on the determined insertion length to read out the corresponding pattern classification. An example of the endoscope capable of determining the insertion length of the insertion section 26 is an endoscope described in JP 2009-77859 A in which the insertion length can be determined by optical sensors arrayed in the insertion section of the endoscope.

The pattern classification corresponding to the diagnostic site may be read out by using these methods in combination or selecting one of them.

The vascular matching and pit matching in the matching section 86 is not limited to the type in which matching with all the images or frames is always performed.

For example when no instruction is issued for vascular matching or pit matching (pattern matching of a special light observation image (diagnosis support by pattern matching)) as in the case of withdrawing the insertion section 26 of the endoscope 12 after the end of the diagnosis, vascular matching and pit matching are of course not be performed as described above.

Alternatively, vascular matching and/or pit matching may only be performed on a still image captured in accordance with an inputted instruction.

Whether to perform pattern matching may be determined depending on the amount of blood vessels or pits extracted from the special light observation image.

In general, the lesion has more microvessels with increasing cancer invasion depth, which often makes the blood stream more complicated. Therefore, the amount or density of blood vessels or pits extracted from the special light observation image in the extracting section 84 may be detected in the matching section 86 or the extracting section 84 so that the matching section 86 can perform vascular matching or pit matching only when the amount of blood vessels or pits exceeds a predetermined threshold.

In this process, the amount of blood vessels or pits may be determined from, for example, the area ratio of blood vessels or pits in the image.

For the same reason as above, vascular matching and pit matching are not limited to cases in which all the extracted blood vessels and pits are checked for matching and vascular matching and pit matching may be performed in an extracted region where the amount of extracted blood vessels and pits is large (or the density is high).

Depending on whether or not the pattern matching is to be performed on an image basis, the region where a large amount of blood vessels and pits are extracted may be determined by the ratio of areas of blood vessels and pits per unit area.

The load on the matching section 86 (image processor 14a) can be reduced by appropriately selecting an image or a region in the image which is to be subjected to vascular matching or pit matching.

In the endoscope system 10 of the invention, the pattern storage section 82 may have a plurality of types of pattern classification for each diagnostic site.

In this case, a selector may be provided in the input device 20 or in the operating section 28 of the endoscope 12 so that vascular matching and pit matching may be performed with a type of pattern classification selected with the selector. Alternatively, all the types of pattern classification or two or more appropriately selected types of pattern classification may be used to perform vascular matching and/or pit matching so that the most reliable result of those obtained by the respective types of pattern classification can be used as the result of vascular matching and/or pit matching. Yet alternatively, the result of vascular matching or pit matching may be derived from the average of the results obtained by using all the types of pattern classification or two or more appropriately selected types of pattern classification.

Selection of a type of pattern classification and processing using a plurality of types of pattern classification may be provided as selectable modes.

The results of vascular matching and pit matching obtained in the matching section 86 (hereinafter referred to simply as “matching results”) are supplied to the support information generating section 90.

The support information generating section 90 sets the diagnosis support for facilitating and ensuring the subsequent observation and diagnosis in accordance with the matching result obtained in the matching section 86 (cancer invasion depth in the vascular patterns) and supplies an instruction for the set diagnosis support to a corresponding site.

Exemplary diagnosis support operations based on the matching results in the endoscope system 10 of the invention include image processing of a special light observation image; automatic adjustment of the imaging conditions (automatic adjustment of the operating conditions of the endoscope 12 and the light source device 16; display (output) of a matching result; and suggestion of preferred imaging conditions. Combinations of a plurality of diagnosis support including display of a matching result followed by suggestion of preferred imaging conditions and suggestion of preferred imaging conditions followed by automatic adjustment of the imaging conditions are also preferred.

One or more than one diagnosis support may be performed. Alternatively, a plurality of diagnosis support (combinations thereof are also included) may be set in the endoscope system 10 so that one or more diagnosis support selected/specified with the selector provided in the input device 20 or the operating section 28 can be performed.

The subsequent processing, action and operation of the endoscope system 10 concerning blood vessels and vascular patterns are basically the same as those concerning pits and pit patterns and therefore in the following description a blood vessel is taken as a typical example and a pit is described or illustrated where appropriate.

An exemplary image processing performed on a special light observation image in accordance with the matching result is enhancement of a blood vessel which matches with a vascular pattern in the pattern classification stored in the pattern storage section 82 (hereinafter referred to simply as “blood vessel matching with a vascular pattern.”

More specifically, upon receipt of the matching result from the matching section 86, the support information generating section 90 supplies to the support image generating portion 78c the shape and position of the blood vessel matching with the vascular pattern and an instruction for enhancement of the blood vessel. The support image generating portion 78c then enhances the blood vessel matching with the vascular pattern on the special light observation image supplied from the image processing portion 78b.

The support image generating portion 78c enhances the special light observation image and supplies the enhanced special observation image (image signals thereof) to the display signal generating section 80. A doctor can thus easily detect a cancerous lesion.

The method of blood vessel enhancement in the support image generating portion 78c is not particularly limited and at least one of density increase, saturation increase, sharpening and differentiation from peripheral image colors by changes in color or coloring may be used. It is also preferred to select an enhancement step to be performed and issue an instruction therefor.

Image processing may be performed by any known method. In addition, the support information generating section 90 may change the intensity of the enhancement in accordance with the cancer invasion depth detected from the matching result.

For example, in the image processing performed, the blood vessel concentration, the blood vessel saturation, the blood vessel sharpness or the color difference between the blood vessel and its periphery is increased with increasing cancer invasion depth, or two or more of the methods are used in combination. A doctor examining the special light observation image can thus easily know the cancer invasion depth.

In such vascular enhancement, the enhancement intensity may be appropriately set in accordance with the invasion depth the vascular pattern indicates.

The automatic adjustment of the imaging conditions in accordance with the matching result is adjusting the imaging conditions in the special light observation so that a lesion on the image can be more easily observed. In other words, this is the feedback control of the endoscope 12 and/or the light source device 16 in accordance with the matching result.

More specifically, examples of such adjustment include one or more of adjustment of quantities of the observation light in the light source device 16, adjustment of the ratio of the quantities of the observation light from the 445LD 60 and 405LD 62, adjustment of the imaging magnification in the endoscope 12, and adjustment of the diaphragm (storage time (shutter speed) of the CCD sensor 48).

The support information generating section 90 sets a method of adjusting the imaging conditions in accordance with the matching result and issues an instruction for the adjustment of the imaging conditions to the endoscope 12 and/or the light source device 16 (controller 14b).

For example, when a blood vessel matching with a vascular pattern, that is, a blood vessel deemed to be affected is detected, a more detailed observation or examination is necessary.

Therefore, in this case, the observation light may be increased to predetermined quantities to increase the image lightness so as to further facilitate the observation and diagnosis. The f-number may be alternatively or simultaneously increased to a predetermined value (the storage time of the CCD sensor 48 is shortened) to increase the image sharpness. In addition, the quantity of light and/or the f-number may be increased with increasing invasion depth that the vascular pattern matching with the blood vessel indicates.

In the illustrated endoscope system 10, microvessels (pits) in the superficial layer can be more clearly observed with increasing ratio of the light quantity of the 405LD 62 to that of the 445LD 60 in the light source device 16.

Therefore, if a blood vessel matching with a vascular pattern is detected, the ratio of the light quantity of the 405LD 62 may be increased to a predetermined value so that microvessels can be more easily observed. The light quantities may be adjusted in the light source device 16 so that the ratio of the light quantity of the 405LD 62 is increased with increasing invasion depth that the vascular pattern matching with the blood vessel indicates.

As described above, in the general diagnosis using the endoscope 12, screening is performed at a low magnification and when an area deemed to be affected is detected, the observation magnification is increased to make a more detailed observation.

Therefore, when a blood vessel matching with a vascular pattern is detected, the imaging magnification in the endoscope 12 may be automatically increased to a predetermined value to enable a more detailed observation. In addition, the observation magnification may be increased with increasing invasion depth that the vascular pattern matching with the blood vessel indicates.

In the automatic adjustment of preferred imaging conditions based on the matching result, the observation light quantities, the ratio of quantities of light from the light sources and the imaging magnification may be appropriately set in accordance with the invasion depth in the vascular patterns.

In the endoscope system 10 of the invention, the imaging conditions used when the respective types of pattern classification were prepared (e.g., quantities of light from the light sources, imaging magnification, f-number and ratio of quantities of light from the light sources) may be stored in association with the pattern classification so that the support information generating section 90 can adjust the imaging conditions in accordance with the imaging conditions used in the preparation of the pattern classification.

More specifically, when a blood vessel matching with a vascular pattern is detected, the imaging conditions may be automatically adjusted so as to be the same as or closer to those used in the preparation of the pattern classification including this vascular pattern.

As described above, in the general diagnosis using the endoscope 12, screening is performed at a low magnification and when an area deemed to be affected is detected, the observation magnification is increased to make a more detailed observation. Therefore, according to this configuration, a blood vessel deemed to be affected can be observed precisely under the same conditions as those of the observation made by a doctor who prepared the pattern classification and is highly experienced in the diagnosis with an endoscope, and hence even a less-experienced doctor can make a correct diagnosis.

When pattern classification to be used is designated, the imaging conditions may be automatically adjusted at the point in time when a blood vessel matching with a vascular pattern is detected so that the imaging conditions can be the same as those used in the preparation of the designated pattern classification although the doctor is made to operate the endoscope at the beginning at his or her own discretion.

Alternatively, when pattern classification to be used is designated, the imaging conditions may be automatically adjusted from the beginning so that the imaging conditions can be the same as those used in the preparation of the designated pattern classification. Yet, alternatively, the imaging conditions may be adjusted in response to an instruction inputted by the doctor for changing the imaging conditions so that the imaging conditions can be the same as those used in the preparation of the designated pattern classification although the doctor is made to operate the endoscope at the beginning at his or her own discretion.

Display of a matching result is to display the matching result obtained in the matching section 86 on the monitor 18.

Instead of display on the monitor 18, the matching result may be outputted by sounding a warning alarm or as voice, or voice output may be used in combination with the display on the monitor 18. The same applies to the suggestion of preferred imaging conditions to be described later.

Assuming, for example, that a blood vessel matching with the type 3I vascular pattern in the pattern classification of The Jikei University School of Medicine was detected as a result of the matching in the matching section 86, the support information generating section 90 issues an instruction to the display signal generating section 80 for displaying a message such as “A blood vessel deemed to match with the type 3I in the pattern classification of The Jikei University School of Medicine was detected.”

Upon receipt of the instruction, the display signal generating section 80 causes the monitor 18 to display the matching result. Even a less-experienced doctor can thus easily detect an affected blood vessel and make a proper diagnosis.

Display (output) of such matching result may be performed only for the matching result obtained in one type of pattern classification or in more than one type of pattern classification.

Suggestion of preferred imaging conditions is to display on the monitor 18 a message for prompting or recommending changes for preferred imaging conditions suitable to the matching result. In the following description, display of a matching result and suggestion of preferred imaging conditions are also collectively referred to as “display for diagnosis support.”

More specifically, the support information generating section 90 does not perform the foregoing automatic adjustment of the imaging conditions but issues to the display signal generating section 80 an instruction for displaying a message such as “Increase the observation light quantities” or “Increase the observation magnification” so as to recommend the doctor to perform the same adjustment of the imaging conditions as the foregoing automatic adjustment of the imaging conditions in accordance with the matching result.

Also in this process, imaging conditions may be suggested so that a larger adjustment is made with increasing cancer invasion depth that the vascular pattern matching with the blood vessel indicates.

In cases where the imaging conditions used in the preparation of each pattern classification are stored as described above, a display for suggesting the imaging conditions to be applied may be made at the point in time when a blood vessel matching with a vascular pattern is detected so that the suggested imaging conditions are those used in the preparation of the pattern classification including this vascular pattern.

An increase in the observation magnification may cause the blood vessel which matched with the vascular pattern, that is, a lesion or an area deemed to be affected to be outside of the field of observation

In such a case, the support information generating section 90 may support the operation of the insertion section 26 of the endoscope 12 for diagnostic purposes by displaying on the monitor 18 a message such as “Move the field of observation to right” or “Move the field of observation upward” so that the affected area is kept within the field of observation or is returned to the field of observation. The diagnosis support on the operation of the insertion section 26 is also available in the case of the foregoing automatic adjustment of the imaging conditions.

In the endoscope system 10 of the invention, the diagnosis support based on the matching result may be appropriately combined and performed.

For example, the display of a matching result may be combined with the suggestion of preferred imaging conditions to display on the monitor 18 a message such as “A blood vessel deemed to be of type 3I in the pattern classification of The Jikei University School of Medicine was detected. Increase the imaging magnification.”

The display of a matching result may be combined with the automatic adjustment of the imaging conditions to automatically improve the imaging conditions such as the observation light quantities and the imaging magnification after a message such as “A blood vessel deemed to be of type 3I in the pattern classification of The Jikei University School of Medicine was detected.” is displayed.

The suggestion of preferred imaging conditions may be combined with the automatic adjustment of the imaging conditions to automatically increase the imaging magnification after a message such as “The imaging magnification is increased.” is displayed. In this combination, a question message such as “Is the imaging magnification increased?” may be displayed so that the imaging magnification may be automatically increased only in cases where the doctor instructed to increase the imaging magnification.

The display of a matching result may be combined with image processing to automatically enhance or display in an enhanced form the blood vessel matching with the vascular pattern after a message such as “A blood vessel deemed to be of type 3I in the pattern classification of The Jikei University School of Medicine was detected.” is displayed.

Also in this case, a question message such as “A blood vessel deemed to be of type 3I in the pattern classification of The Jikei University School of Medicine was detected. Is the display enhanced?” may be displayed so that the blood vessel is enhanced only in cases where the doctor instructed to increase the imaging magnification.

The display of a matching result may be combined with the suggestion of preferred imaging conditions and the automatic adjustment of the imaging conditions to automatically increase the imaging magnification after a message such as “A blood vessel deemed to be of type 3I in the pattern classification of The Jikei University School of Medicine was detected. The imaging magnification is increased.” is displayed.

In addition, when the imaging conditions used in the preparation of each pattern classification are stored, the imaging conditions may be changed after a message such as “A blood vessel deemed to be of type 3I in the pattern classification of The Jikei University School of Medicine was detected. The imaging conditions are changed according to the pattern classification in The Jikei University School of Medicine.” is displayed.

Also in these cases, a question message may be displayed as described above so that the imaging conditions can be changed according to the instruction from the doctor.

When the imaging conditions used in the preparation of each pattern classification are stored, the imaging conditions may be changed after a message such as “The imaging conditions are changed to conform with the pattern classification in The Jikei University School of Medicine” is displayed at the point in time when the pattern classification to be used is designated. Also in this case, a question message may be displayed as described above so that the imaging conditions can be changed according to the instruction from the doctor.

In the case of a subject who has an experience that a blood vessel matching with a vascular pattern was detected, the imaging conditions may be automatically set so as to be the same as the previous imaging conditions. Also in this case, the imaging conditions may be changed after a message which suggests setting the imaging conditions is displayed or according to the instruction from the doctor who read the question message as described above.

The display signal generating section 80 subjects the normal light observation image supplied from the normal light image generating section 76 (image processing portion 76b) or the special light observation image supplied from the special light image generating section 78 (image processing portion 78b or the support image generating portion 78c) to color space conversion, scaling and other necessary processing steps, or image allocation, incorporation of character information such as the name of a subject and other necessary processing steps to generate a display image having the composite image incorporated therein and this image is displayed on the monitor 18.

When an instruction is given to display the diagnosis support suitable to the matching result, the display signal generating section 80 generates a display image which includes character information for the diagnosis support and causes the monitor 18 to display the image.

In the endoscope system 10, images are displayed on the monitor 18 in various ways, as exemplified by the display of only the special light observation image, the display of only the normal light observation image, the display of only the image enhanced based on the matching result, the display of the normal light observation image and the special light observation image, the display of the special light observation image and the image enhanced based on the matching result, the display of the normal light observation image and the image enhanced based on the matching result, the display of the normal light observation image, the special light observation image and the image enhanced based on the matching result, and the toggle display of two or more of the normal light observation image, the special light observation image and the image enhanced based on the matching result according to the switching instruction.

Therefore, at least two of them are preferably provided as selectable modes.

The display signal generating section 80 generates an image suitable to the selected display mode and causes the monitor 18 to display the generated image. In addition, when more than one monitor 18 is used or when there is enough space in the display region, the respective vascular patterns of the vascular pattern classification may be displayed for reference.

When an instruction is given to display the image enhanced based on the matching result, image processing (vascular enhancement) of the special light observation image based on the matching result can be selected according to the instruction.

When an instruction is given to display the diagnosis support, the display signal generating section 80 generates a display image for the display of the diagnosis support based on the matching result irrespective of which display mode is selected.

An example of the operation of the endoscope system 10 is described below for the case of the special light observation.

When the input device 20 issues an instruction for the start of imaging with the endoscope 12, the 445LD 60 and the 405LD 62 of the light source device 16 are turned on. The CCD sensor 48 of the endoscope 12 starts imaging (photometry).

B light (B laser beam) emitted from the 445LD 60 and BV light (BV laser beam) emitted from the 405LD 62 are multiplexed by the multiplexer 64, propagated through the optical fiber 68 and supplied from the connecting portion 16a to the connector 32 of the endoscope 12 as observation light (excitation light).

The observation light supplied to the connector 32 of the endoscope 12 propagates through the optical fiber 52 to the scope portion 42, where the observation light is emitted from the distal end of the optical fiber 52 to enter the fluorescent body 54.

The B light emitted from the 445LD 60 excites the fluorescent body 54 which emits green to yellow excitation light. The excitation light is combined with part of the B light which passed through the fluorescent body 54 to form pseudo white light, which serves as observation light and is irradiated through the illumination lens 50 on the observation site in the living body. Almost all the BV light emitted from the 405LD 62 passes through the fluorescent body 54 to be irradiated through the illumination lens 50 on the observation site as narrowband BV observation light for use in the special light observation.

The observation site having received the observation light is imaged through the imaging lens 46 on the light-receiving surface of the CCD sensor 48, which captures an R image, a G image and a B image (performs photometry).

Output signals from the CCD sensor 48 are supplied to the AFE board 56. The AFE board 56 subjects the output signals from the CCD sensor 48 to noise removal by correlated double sampling, amplification and A/D conversion to obtain digital image signals, which are then supplied to the DSP 72 of the processing device 14 (processor 14a).

The DSP 72 subjects the supplied image (image signals) to predetermined processing such as gamma correction and color correction and stores the processed image in a predetermined portion of the image storage section 74.

Once the image signals are stored in the image storage section 74, the read-out portion 78a of the special light image generating section 78 reads out the narrowband BV image and the G image from the image storage section 74 and supplies them to the image processing portion 78b.

The image processing portion 78b having received the narrowband BV image and the G image allocates the G image to R pixels to be displayed and the narrowband BV image to B and G pixels to be displayed to form pixels each composed of three sub-pixels, and the image is further subjected to color conversion, color enhancement and image structure enhancement to obtain the special light observation image.

In this example, the image processing portion 78b supplies to the extracting section 84 and the support image generating portion 78c the generated special light observation image for which instructions were given to display the diagnosis support and enhance a blood vessel matching with a vascular pattern.

The extracting section 84 analyzes the special light observation image supplied thereto, for example, through binarization to extract blood vessels or pits, and supplies the extracted blood vessels or pits to the matching section 86.

The matching section 86 having received the blood vessels extracted in the extracting section 84 reads out vascular pattern classification from the pattern storage section 82 and performs pattern matching between the blood vessels and the vascular patterns. When the vascular pattern classification to be used is preliminarily designated, the designated vascular pattern classification is read out to perform matching with the blood vessels.

The matching section 86 supplies the matching result to the support information generating section 90.

Assuming in this example that a blood vessel matching with the type 3I vascular pattern in the pattern classification of The Jikei University School of Medicine is detected, the support information generating section 90 having received the matching result issues an instruction to the display signal generating section 80 based on the matching result so that a message such as “A blood vessel deemed to be of type 3I in the pattern classification of The Jikei University School of Medicine was detected. Is the imaging magnification increased?” is displayed for the diagnosis support.

Together with the positional information of the blood vessel, the support information generating section 90 issues an instruction to the support image generating portion 78c so as to enhance the blood vessel matching with the vascular pattern by increasing its density and saturation.

The support image generating portion 78c having received the instruction from the support information generating section 90 accordingly performs enhancement by increasing the density and saturation of the corresponding blood vessels in the special light observation image. After the end of the image processing, the enhanced special light observation image is sent to the display signal generating section 80.

Upon receipt of the enhanced special light observation image together with the instruction message for displaying the diagnosis support “A blood vessel deemed to be of type 3I in the pattern classification of The Jikei University School of Medicine was detected. Is the imaging magnification increased?”, the display signal generating section 80 generates a display image including the enhanced special light observation image and the display of the diagnosis support and causes the monitor 18 to display this image.

Upon receipt of the instruction for increasing the imaging magnification from the doctor, the support information generating section 90 issues an instruction to the endoscope 12 to increase the imaging magnification to a value corresponding to the invasion depth of the type 3I vascular pattern in the pattern classification of The Jikei University School of Medicine.

As is clear from the foregoing description, the invention extracts blood vessels (pits) from an image captured with the endoscope 12, performs matching between the blood vessels and vascular patterns in the preliminarily stored vascular pattern classification, and performs, based on the matching result (cancer invasion depth associated with the vascular patterns), enhancement of a blood vessel matching with a vascular pattern (i.e., affected area), automatic adjustment for preferred imaging conditions such as increased imaging magnification and observation light quantities, and display of the diagnosis support such as suggestion of the matching result and preferred imaging conditions. Therefore, even a less-experienced doctor can correctly diagnose the cancer invasion depth.

While the endoscope system of the invention has been described above in detail, the invention is by no means limited to the above embodiments, and various improvements and modifications may of course be made without departing from the spirit of the invention.

For example, the endoscope system of the invention can also be advantageously used in an endoscope system such as the one described in JP 3559755 B which captures images with a rotary filter and a monochrome CCD sensor in a frame sequential manner.

In other words, the endoscope system of the invention can be used in various known endoscope systems as long as matching of the blood vessels and pits with vascular patterns and pit patterns associated with the invasion depth, and the diagnosis support based on the matching results are performed.

What is claimed is:

1. An endoscope system comprising: an endoscope having an imaging device for photoelectrically capturing an image; a light source device for supplying to said endoscope observation light used for capturing the image in said endoscope; an image processing device for processing the image captured by said endoscope to generate a display image; and a monitor for displaying the display image generated in said image processing device,
wherein said image processing device comprises a storage section for storing at least one of vascular pattern classification including vascular patterns associated with a disease invasion depth and pit pattern classification including pit patterns associated with the disease invasion depth; an extracting section for extracting at least one of blood vessels and pits from the image captured by said endoscope; a matching section for performing pattern matching between at least one of the blood vessels and pits extracted by said extracting section and at least one of the vascular patterns in the vascular pattern classification and the pit patterns in the pit pattern classification which are stored in said storage section; and a diagnosis support section for receiving a result of the pattern matching from said matching section and causing at least one of said endoscope, said light source device, said monitor and said image processing device to perform support operations for diagnosis support based on the result of the pattern matching.

2. The endoscope system according to claim 1, wherein said light source device has a function of supplying narrowband light for special light observation to said endoscope.

3. The endoscope system according to claim 2, wherein said support operations include at least one of adjustment of imaging conditions, adjustment of image processing performed in said image processing device and the diagnosis support through display on said monitor.

4. The endoscope system according to claim 3, wherein the adjustment of the imaging conditions includes at least one of changing an imaging magnification in said endoscope, changing an f-number in said endoscope, adjusting quantities of the observation light from said light source device, and adjusting a spectral distribution of the observation light from said light source device.

5. The endoscope system according to claim 3, wherein the adjustment of the image processing performed in said image processing device includes at least one of enhancement of a blood vessel matching with a vascular pattern in the vascular pattern classification stored in said storage section and enhancement of a pit matching with a pit pattern in the pit pattern classification stored in said storage section.

6. The endoscope system according to claim 3, wherein the diagnosis support through the display on said monitor is the display of the result of the pattern matching in said matching section.

7. The endoscope system according to claim 3, wherein the diagnosis support through the display on said monitor is the display prompting an operator to perform at least one of changing an imaging magnification in said endoscope, changing an f-number in said endoscope, adjusting quantities of the observation light from said light source device, and adjusting a spectral distribution of the observation light from said light source device.

8. The endoscope system according to claim 2, wherein a degree of said support operations is changed according to at least one of the disease invasion depth in a vascular pattern which matched with a blood vessel and the disease invasion depth in a pit pattern which matched with a pit.

9. The endoscope system according to claim 2, further comprising a selector for selecting execution of extraction in said extracting section and execution of the pattern matching in said matching section.

10. The endoscope system according to claim 2, wherein said matching section performs said pattern matching according to at least one of an amount of the blood vessels and an amount of the pits extracted by said extracting section.

11. The endoscope system according to claim 2, wherein said storage section stores at least one of said vascular pattern classification and said pit pattern classification for each of at least two diagnostic sites.

12. The endoscope system according to claim 2 further comprising an indicator for indicating a site to be observed with said endoscope.
